(19) United States
(12) Patent Application Publication
(10) Pub. No.: US 2005/0129622 A1
(43) Pub. Date: Jun. 16, 2005

(54) Nasal composition comprising a mucopolysaccharide and propylene glycol

(76) Inventors: Isabelle Rault, Segny (FR); Giovanna Marzano, Prangins (CH); Urbano Salvi, Nyon (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/518,862

(22) PCT Filed: Jun. 18, 2003
(86) PCT No.: PCT/EP03/06478

(30) Foreign Application Priority Data
Jun. 20, 2002 (EP) 02013693.3

(51) Int. Cl. A61L 9/04; A61K 31/137; A61K 31/737; A61K 31/728; A61K 31/496; A61K 31/727; A61K 31/715

(52) U.S. Cl. 424/45; 514/54; 514/56; 514/649; 514/253.04

(57) ABSTRACT

The invention relates to pharmaceutical compositions adapted to nasal administration. The nasal formulations of the invention are characterized inter alia by having excellent moisturizing properties and not requiring a preservative.

The present invention relates to pharmaceutical compositions intended for nasal administration. More specifically, it concerns nasal formulations with improved moisturizing properties. What is in particular strived for is nasal compositions that further can be formulated “preservative-free”, which means that they do not contain any special preservative and nevertheless fulfill all requirements with respect to microbiological stability, i.e. that germs are killed efficaciously over the whole shelf life of the nasal product concerned.

The nasal administration of active substances is a widely used method of treatment. Active substances that come into consideration are, for example, vasoconstrictors, such as xylometazoline, or antiallergic agents, such as H₁ receptor antagonists, e.g. dimethindene maleate. Another group of possible active substances is e.g. corticosteroids, such as beclomethasone or fluticasone.

The indications in which a certain nasally administered drug is to be applied are known in the art. For example, vasoconstrictors are e.g. used as nasal decongestants for alleviating the typical symptoms of common cold, like running nose, obstructed nose etc., or in rhinitis or sinusitis. Antiallergic agents and corticosteroids are e.g. used in antiallergic conditions, e.g. hay fever, or in anti-asthmatic or anti-inflammatory conditions.

Nasal administration of active substances can be accomplished e.g. by nasal formulations in liquid form, such as drops, solutions, sprays (nebulizers) or metered-dose sprays, or in semi-solid form, such as gels or creams.

However, upon administration of nasal formulations often the patients are suffering from side effects like burning, dryness, stinging of the nasal mucosa or sneezing. One of the main reasons for this is that the nasal mucosa is not sufficiently moisturized and/or is not kept moisturized long enough after administration.

The present invention addresses these problems and provides nasal formulations that exhibit excellent moisturizing properties. Moreover, they can be formulated “preservative-free”. Said goals have been achieved by selecting a specific beneficial mixture of ingredients for said nasal formulations. More concretely, the gist of the present invention lies in combining a mucopolysaccharide with propylene glycol in a nasal formulation and thus obtaining a nasal formulation with unique beneficial properties.

Although the focus in the beginning was primarily on obtaining preservative-free formulations, in the course of experimentations it has been found that said formulations are also very suitable when combined with a preservative. Thus, it is justified to define preservatives as an optional component of the compositions of the invention, with the compositions without preservative being preferred.

The invention therefore relates to a nasal pharmaceutical composition that comprises
(a) at least one active substance suitable for nasal administration,
(b) a mucopolysaccharide, and
(c) propylene glycol.

Active substances suitable for nasal administration (a) are e.g. vasoconstrictors, e.g. xylometazoline, e.g. xylometazoline hydrochloride; indanazoline, metizoline; naphazoline, e.g. naphazoline hydrochloride; fenoxazoline, e.g. fenoxazoline hydrochloride; oxymetazoline, e.g. oxymetazoline hydrochloride; tetrahydrozoline, tramazoline, tymazoline; phenylephrine, e.g. phenylephrine hydrochloride; ephedrine, e.g. d-pseudoephedrine hydrochloride; or epinephrine; or antiallergic agents, such as H₁ receptor antagonists, e.g. dimethindene or a nasally acceptable salt thereof, e.g. dimethindene maleate; acrivastine, brompheniramine, chlorpheniramine, dexchlorpheniramine, triprolidine, bromodiphenhydramine, clemastine, phenyltoloxamine, piprinhydrinate, pyrilamine, tripelennamine, cetirizine, hydroxyzine, methdilazine, promethazine, trimeprazine, azatadine, cyproheptadine, loratadine, astemizole, diphenhydramine, levocabastine or terfenadine. Examples for corticosteroids are e.g. beclomethasone, e.g. beclomethasone dipropionate, or fluticasone, e.g. fluticasone propionate. All active substances which are capable of salt formation may be present either in free form or in the form of a nasally acceptable salt. Also mixtures of more than one active substance come into consideration, e.g. a combination of a vasoconstrictor and an antiallergic agent, such as xylometazoline plus dimethindene or phenylephrine plus dimethindene, or a combination of a vasoconstrictor and a corticosteroid, such as xylometazoline plus beclomethasone.

In one embodiment of the invention, the active substances used are vasoconstrictors, e.g. xylometazoline, naphazoline, fenoxazoline, oxymetazoline, tetrahydrozoline, tramazoline, phenylephrine, ephedrine or epinephrine, or any nasally acceptable salt thereof. In particular preferred are xylometazoline and oxymetazoline, especially xylometazoline, and nasally acceptable salts thereof.

The concentration of the active substances is typically chosen so that a pharmaceutically, i.e. nasally, effective dose thereof can be administered easily, e.g. by a certain number of drops or by spraying.

For example, if a vasoconstrictor is used as active substance (a), it is e.g. present in an amount of from 0.005 up to 0.5%, preferably of from 0.01 up to 0.3%, and in particular of from 0.025 up to 0.2% (w/w) of the total composition.

The term mucopolysaccharide (b) comprises glycosaminoglycans, e.g. heparinoids, e.g. chondroitin, dermatan and nasally acceptable salts of any of said compounds, especially chondroitin sulfate and dermatan sulfate; hyaluronic acid, or a nasally acceptable salt thereof, e.g. sodium hyaluronate; keratan, or a nasally acceptable salt thereof, e.g. keratan sulfate; heparin, or a nasally acceptable salt thereof, e.g. heparin sulfate; or acemannan.

Preferred are chondroitin, or a nasally acceptable salt thereof, e.g. chondroitin sulfate, hyaluronic acid, or a nasally acceptable salt thereof, e.g. sodium hyaluronate; and dermatan, or a nasally acceptable salt thereof, e.g. dermatan sulfate. Especially preferred is chondroitin sulfate.

The component (b) is e.g. present in an amount of from 0.01 up to 5%, preferably of from 0.02 up to 3%, and in particular of from 0.05 up to 2%, (w/w) of the total composition.

Depending on what type of nasal composition is intended (liquid, viscous liquid, gel) the amount of (b) must be adjusted accordingly. Concretely, the more viscous the composition is to be, the more of (b) has typically to be included. The amount of (b) further depends on the kind of mucopolysaccharide (b) used.

Preferred amounts of chondroitin, or a nasally acceptable salt thereof, to be used are of from 0.1 up to 5%, in particular of from 0.25 up to 2%. Preferred amounts of hyaluronic acid, or a nasally acceptable salt thereof, to be used are of from 0.02 up to 1%, in particular of from 0.05 up to 0.5%.

In the nasal compositions of the invention, propylene glycol (c) is typically present in an amount of 0.5 up to 10%, preferably 1 up to 5%, more preferably 1.5 up to 3%, and in particular 1.7 up to 2.5%.

Optionally, the nasal compositions of the invention may further include a nasally acceptable film-forming agent. By adding it, the moisturizing and soothing effects of the compositions of the invention may be reinforced, namely by restricting the loss of water and thus longer maintaining a good level of hydration of the nasal mucosa. That way the comfort sensation of the patient may further be improved. Preferred are water soluble or swellable cellulose materials, e.g. hydroxypropyl methyl cellulose, hydroxypropyl cellulose, methyl cellulose, hydroxyethyl methyl cellulose or sodium carboxymethyl cellulose, and polyvinylpyrrolidone (povidone) or cross-linked polyvinylpyrrolidone (crospovidone).

Optionally, the nasal compositions of the invention may further include a nasally acceptable preservative. The latter are well known in the art. Examples are benzalkonium chloride, benzoxonium chloride, benzododecinium bromide, benzethonium chloride, cetylpyridinium chloride, cetrimide; benzoic acid and esters and salts thereof, e.g. C1-C7-alkyl esters of 4-hydroxybenzoic acid, such as methyl 4-hydroxybenzoate, sodium methyl 4-hydroxybenzoate or propyl 4-hydroxybenzoate; chlorhexidine or nasally acceptable salts thereof, e.g. chlorhexidine digluconate, chlorhexidine acetate or chlorhexidine chloride; 2-phenylethanol, 2-phenoxyethanol and sorbic acid. If present, they are used in usual amounts, e.g. benzalkonium chloride and benzoxonium chloride typically in amounts of from 0.005 up to 0.03%, in particular 0.01-0.02%, (w/w) of the total composition.

In another embodiment of the invention, the nasal compositions of the invention are devoid of an additional nasally acceptable preservative.

Optionally, the nasal compositions of the invention may further include an essential oil of a plant, e.g. lavender, rosemary or tea tree, especially in the form of a water-soluble extract. vehicle is usually present in an amount of at least 90%—preferably at least 92%, especially at least 94% and in particular at least 96%—(w/w) of the total composition. The vehicle is typically water.

Moreover, the nasal compositions of the invention may contain usual nasally acceptable excipients that are known in the art and include e.g. buffering agents, chelating agents, precipitation inhibitiors (e.g. glycine) and/or isotonicity regulators. Typically, they do not include any phospholipids. Typically, they are devoid of a polycarbophil (polycarbophils are polymers of acrylic acid crosslinked with pblyalkenyl ethers or divinyl glycol). More typically, the nasal compositions of the invention are devoid of both a polycarbophil and polyvinyl alcohol. Even more typically, they are devoid of both phospholipids and a polycarbophil. Most typically, they are devoid of all of phospholipids, a polycarbophil and polyvinyl alcohol.

In a further embodiment of the invention, the nasal compositions may include any at least one active substance suitable for nasal administration as defined hereinbefore and hereinafter but they are devoid of fexofenadine and pharmaceutically acceptable salts thereof.

The nasal compositions of the invention show e.g. excellent moisturizing and soothing properties, they cause a sensation of comfort, and therefore test persons excellently accept them. A significant reduction of symptoms like burning, dryness, stinging of the nasal mucosa or sneezing is found upon administration of the compositions.

The beneficial properties of the compositions of the invention can be demonstrated e.g. by the following tests: For example, the moisturizing properties can be shown in hair humidity measurements by transient thermal transfer, e.g. in the Hydrascane device provided by Laboratoire Dermscan, France. Or the level of hydration of the nasal mucosa can also be demonstrated e.g. by showing the distribution of tritiated water within a mucosa model, e.g. pig trachea. In microbiological “challenge” tests, e.g. over 6 weeks, the compositions of the invention—including those comprising no special preservative—remain free of germs.

Moreover, consumer research studies show that the nasal compositions of the invention, surprisingly, are perceived more moisturizing and less drying than other commercially available compositions.

The nasal compositions of the invention can be manufactured in a manner known per se, for example by conventional mixing and dissolution methods in aqueous vehicles. Typically, they are filled in containers known per se for the storage and application of nasal compositions, e.g. metered-dose spray devices, devices for sprays, squeeze bottles or bottles for drops.

The following examples illustrate the invention.

### EXAMPLE 1

### Nasal Spray Composition Containing 0.1% (w/w) of Xylometazoline Hydrochloride

| Ingredients | Amount (kg/100 kg) |
|---|---|
| Xylometazoline hydrochloride | 0.10 |
| Chondroitin sulfate | 1.0 |
| Propylene glycol | 2.0 |
| Sodium dihydrogen phosphate dihydrate | 0.16 |
| Disodium phosphate dodecahydrate | 0.085 |
| Disodium edetate | 0.05 |
| Purified water | ad 100.0 |

Manufacturing method (for a batch of 100 liters): Introduce 88.605 kg of purified water into a dissolutor, add chondroitin sulfate under stirring and continue to stir until dissolution will be complete. Add sodium dihydrogen phosphate dihydrate, disodium phosphate dodecahydrate, disodium edetate and stir until complete dissolution. Add propylene glycol under stirring and xylometazoline hydrochloride to the solution, continue to stir until dissolution will be complete. Rinse with 8.0 kg of purified water. Filter solution through a 0.22 micrometer filter.

### EXAMPLE 1a

Nasal spray composition containing 0.05% (w/w) of xvlometazoline hydrochloride is manufactured analogously to Example 1 by using 0.05 kg of xylometazoline hydrochloride (instead of 0.10 kg) and starting with 88.655 kg of purified water (instead of 88.605 kg).

### EXAMPLE 2

### Nasal Spray Composition Containing 0.1% (w/w) of Xylometazoline Hydrochloride (with Film-Forming Agent)

| Ingredients | Amount (kg/100 kg) |
|---|---|
| (a) Xylometazoline hydrochloride | 0.10 |
| (b) Chondroitin sulfate | 1.0 |
| (c) Propylene glycol | 2.0 |
| (d) Sodium dihydrogen phosphate dihydrate | 0.16 |
| (e) Disodium phosphate dodecahydrate | 0.085 |
| (f) Disodium edetate | 0.05 |
| (g) Hydroxypropyl methyl cellulose | 0.10 |
| (h) Purified water | ad 100.0 |

Manufacturing method (for a batch of 100 liters): Introduce 88.505 kg of h into a dissolutor, disperse g under stirring, and after dissolution continue to stir for 30 minutes. Add b under stirring and continue to stir until dissolution will be complete. Add d, e, f and stir until complete dissolution. Add c under stirring and a to the solution. Continue to stir until dissolution of a will be complete. Rince with 8.0 kg of h. Filter solution through a 0.22 micrometer filter.

### EXAMPLE 2a:

Nasal spray composition containing 0.05% (w/w) of xviometazoline hydrochloride (with film-forming agent) is manufactured analogously to Example 2 by using 0.05 kg of xylometazoline hydrochloride (instead of 0.10 kg) and starting with 88.555 kg of purified water (instead of 88.505 kg).

### EXAMPLE 3

### Nasal Spray Composition Containing 0.1% (w/w) of Xylometazoline Hydrochloride

| Ingredients | Amount (kg/100 kg) |
|---|---|
| Xylometazoline hydrochloride | 0.10 |
| Sodium hyaluronate | 0.10 |
| Propylene glycol | 2.0 |
| Sodium dihydrogen phosphate dihydrate | 0.16 |
| Disodium phosphate dodecahydrate | 0.085 |
| Disodium edetate | 0.05 |
| Purified water | ad 100.0 |

Manufacture is analogous to Example 1.

### EXAMPLE 4

### Nasal Spray Composition Containing 0.1% (w/w) of Xylometazoline Hydrochloride (with Lavender Essential Oil)

| | Amount |
|---|---|
| Ingredients | (kg/100 kg) |
| (a) Xylometazoline hydrochloride | 0.10 |
| (b) Chondroitin sulfate | 1.0 |
| (c) Propylene glycol | 2.0 |
| (d) Sodium dihydrogen phosphate dihydrate | 0.16 |
| (e) Disodium phosphate dodecahydrate | 0.085 |
| (f) Disodium edetate | 0.05 |
| (g) Hydroxypropyl methyl cellulose | 0.10 |
| (h) Lavender essential oil | 0.10 |
| (i) Cremophor RH40 (=PEG-40 hydrogenated castor oil) | 0.50 |
| (j) Purified water | ad 100.0 |

Manufacturing method (for a batch of 100 liters): Introduce 87.905 kg of j into a dissolutor, disperse g under stirring, and after dissolution continue to stir for 30 minutes. Add b under stirring and continue to stir until dissolution will be complete. Add d, e, f and stir until complete dissolution. Add c under stirring and a to the solution. Continue to stir until dissolution of a will be complete. Introduce into a small stainless steel container i, add h and stir until a clear solution is obtained. Then slowly add 8.0 kg of j. Introduce said latter solution into the former one. Filter combined solution through a 0.22 micrometer filter.

### EXAMPLE 4a

Nasal spray composition containing 0.1% (w/w) of xvlometazoline hydrochloride (with tea tree essential oil) is manufactured analogously to Example 4 by using 0.10 kg of tea tree oil (instead of 0.10 kg of lavender oil).

### EXAMPLE 5

### Nasal Drop Composition Containing 0.1% (w/w) of Xylometazoline Hydrochloride (with Preservative Chlorhexidine Digluconate)

| Ingredients | Amount (kg/100 kg) |
|---|---|
| (a) Xylometazoline hydrochloride | 0.10 |
| (b) Chondroitin sulfate | 1.0 |
| (c) Propylene glycol | 2.0 |
| (d) Disodium edetate | 0.05 |
| (e) Hydroxypropyl methyl cellulose | 0.10 |
| (f) Citric acid | 0.10 |
| (g) Disodium phosphate anhydous | 0.22 |
| (h) Chlorhexidine digluconate | 0.02 |
| (i) Purified water | ad 100.0 |

Manufacturing method (for a batch of 100 liters): Introduce 96.41 kg of i into a dissolutor, disperse e under stirring, and after dissolution continue to stir for 30 minutes. Add g and f under stirring until dissolution, then add b and continue to stir until dissolution will be complete. Maintain stirring for further 15 minutes. Dissolve d, h, c and a in the solution. Continue to stir until dissolution of a will be complete. Filter solution through a 0.45 micrometer filter.

### EXAMPLE 5a

Nasal spray composition containing 0.1% (w/w) of Xylometazoline hydrochloride (with preservative cetylpyridinium chloride) is manufactured analogously to Example 5 by using 0.02 kg of cetylpyridinium chloride (instead of 0.02 kg of chlorhexidine digluconate).

### EXAMPLE 5b

Nasal drop composition containing 0.1% (w/w) of xylometazoline hydrochloride (with preservative benzoxonium chloride) is manufactured analogously to Example 5 by using 0.02 kg of benzoxonium chloride (instead of 0.02 kg of chlorhexidine digluconate).

### EXAMPLE 5c

Nasal drop composition containing 0.1% (w/w) of xvlometazoline hydrochloride (with preservative benzalkonium chloride) is manufactured analogously to Example 5 by using 0.02 kg of benzalkonium chloride (instead of 0.02 kg of chlorhexidine digluconate).

### EXAMPLE 6

### Nasal Drop Composition Containing 0.1% (w/w) of Xylometazoline Hydrochloride (with Preservative Methyl 4-Hydroxybenzoate)

| Ingredients | Amount (kg/100 kg) |
|---|---|
| (a) Xylometazoline hydrochloride | 0.10 |
| (b) Chondroitin sulfate | 1.0 |
| (c) Propylene glycol | 2.0 |
| (d) Sodium dihydrogen phosphate dihydrate | 0.16 |
| (e) Disodium phosphate dodecahydrate | 0.085 |
| (f) Disodium edetate | 0.05 |
| (g) Hydroxypropyl methyl cellulose | 0.10 |
| (h) Methyl 4-hydroxybenzoate | 0.15 |
| (i) Purified water | ad 100.0 |

Manufacturing method (for a batch of 100 liters): Introduce 96.355 kg of i into a dissolutor and heat to 85° C., add h and maintain at this temperature under stirring for about 15 minutes until complete dissolution. Cool down to 75° C. and add d and e. Continue to cool down to 35° C., then disperse g under stirring, and—after dissolution—continue to stir for 30 minutes. Add b and continue to stir until dissolution will be complete. Maintain the stirring for further 15 minutes. Dissolve f, c and a in the solution. Continue to stir until dissolution of a will be complete. Filter solution through a 0.45 micrometer filter.

### EXAMPLE 7

Nasal spray composition containing 0.05% (w/w) of Oxymetazoline hydrochloride is manufactured in a manner analogous to Example la by using 0.05 kg of oxymetazoline hydrochloride (instead of 0.05 kg of xylometazoline hydrochloride).

### EXAMPLE 8

Nasal spray composition containing 0.1% (w/w) of Oxymetazoline hydrochloride is manufactured in a manner analogous to Example 1 by using 0.10 kg of oxymetazoline hydrochloride (instead of 0.10 kg of xylometazoline hydrochloride).

### EXAMPLE 9

Nasal Spray Composition Containing 0.1% (w/w) of Xylometazoline Hydrochloride

| Ingredients | Amount (kg/100 kg) |
|---|---|
| Xylometazoline hydrochloride | 0.10 |
| Chondroitin sulfate (Injectable grade) | 1.5 |
| Propylene glycol | 2.3 |
| Sodium dihydrogen phosphate dihydrate | 0.16 |
| Disodium phosphate dodecahydrate | 0.085 |
| Disodium edetate | 0.05 |
| Purified water | ad 100.0 |

Manufacture is analogous to Example 1.

### EXAMPLE 10

### Nasal Spray Composition Containing 0.1% (w/w) of Xylometazoline Hydrochloride

| Ingredients | Amount (kg/100 kg) |
|---|---|
| Xylometazoline hydrochloride | 0.10 |
| Chondroitin sulfate (Injectable grade) | 1.0 |
| Propylene glycol | 2.0 |
| Citric acid monohydrate | 0.05 |
| Sodium citrate | 0.26 |
| Purified water | ad 100.0 |

Manufacture is analogous to Example 1 (citrate buffer is added instead of phosphate buffer).

### EXAMPLE 11

### Nasal Drop Composition Containing 0.1% (w/w) of Xylometazoline Hydrochloride (with Preservative 2-Phenylethanol)

| Ingredients | Amount (kg/100 kg) |
|---|---|
| (a) Xylometazoline hydrochloride | 0.10 |
| (b) Chondroitin sulfate (Injectable grade) | 1.0 |
| (c) Propylene glycol | 2.0 |
| (d) Sodium dihydrogen phosphate dihydrate | 0.16 |
| (e) Disodium phosphate dodecahydrate | 0.085 |
| (f) Disodium edetate | 0.05 |
| (g) 2-Phenylethanol | 0.45 |
| (h) Purified water | ad 100.0 |

Manufacturing method (for a batch of 100 liters): Introduce 96.155 kg of h into a dissolutor, add b under stirring and continue to stir until dissolution will be complete. Add d, e, f and stir until complete dissolution. Add c under stirring, then g and a to the solution and continue to stir until dissolution will be complete. Filter solution through a 0.45 micrometer filter.

### EXAMPLE 11a

Nasal drop composition containing 0.1% (w/w) of Xylometazoline hydrochloride (with preservative 2-phenoxyethanol) is manufactured analogously to Example 11 by using 0.45 kg of 2-phenoxyethanol (instead of 0.45 kg 2-phenylethanol).

### EXAMPLE 12

### Nasal Drop Composition Containing 0.1% (w/w) of Xylometazoline Hydrochloride (with Preservative Sodium Methyl 4-Hydroxybenzoate)

| Ingredients | Amount (kg/100 kg) |
|---|---|
| (a) Xylometazoline hydrochloride | 0.10 |
| (b) Chondroitin sulfate (Injectable grade) | 1.0 |
| (c) Propylene glycol | 2.0 |
| (d) Sodium dihydrogen phosphate dihydrate | 0.16 |
| (e) Disodium phosphate dodecahydrate | 0.085 |
| (f) Disodium edetate | 0.05 |
| (g) Methyl 4-hydroxybenzoate, sodium salt | 0.12 |
| (h) Purified water | ad 100.0 |

Manufacturing method (for a batch of 100 liters): Introduce 96.485 kg of h into a dissolutor, add b under stirring and continue to stir until dissolution will be complete. Add d, e, f and stir until complete dissolution. Add c under stirring, then g and a to the solution and continue to stir until dissolution will be complete. Filter solution through a 0.45 micrometer filter.

### EXAMPLE 13

### Nasal Drop Composition Containing 0.1% (w/w) of Xylometazoline Hydrochloride (with Preservative Methyl 4-Hydroxybenzoate)

| Ingredients | Amount (kg/100 kg) |
|---|---|
| (a) Xylometazoline hydrochloride | 0.1 |
| (b) Chondroitin sulfate | 0.5 |
| (c) Propylene glycol | 1.8 |
| (d) Sodium dihydrogen phosphate dihydrate | 0.16 |
| (e) Disodium phosphate dodecahydrate | 0.085 |
| (f) Disodium edetate | 0.05 |
| (g) Methyl 4-hydroxybenzoate | 0.12 |
| (h) Purified water | ad 100.0 |

Manufacturing method (for a batch of 100 liters): Introduce into a dissolutor 97.185 kg of h and heat to 85° C., add g and maintain under stirring at this temperature for about 15 minutes until complete dissolution. Cool down to 75° C. and add d and e. Continue to cool down to 35° C. Add b, continue to stir until dissolution will be complete and stir for further 15 minutes. Add f, c and a to the solution. Continue to stir until dissolution of a will be complete. Filter solution through a 0.45 micrometer filter.

### EXAMPLE 13a

Nasal drop composition containing 0.1% (w/w) of Xylometazoline hydrochloride (with preservatives methyl 4-hydroxybenzoate and propyl 4-hydroxybenzoate) are manufactured analogously to Example 13 by using 0.075 kg of methyl 4-hydroxy-benzoate and 0.025 kg of propyl 4-hydroxybenzoate (instead of 0.12 kg methyl 4-hydroxybenzoate).

### EXAMPLE 14

### Nasal Drop Composition Containing 0.1% (w/w) of Xylometazoline Hydrochloride (with Preservative Sorbic Acid)

| Ingredients | Amount (kg/100 kg) |
|---|---|
| (a) Xylometazoline hydrochloride | 0.10 |
| (b) Chondroitin sulfate (Injectable grade) | 1.0 |
| (c) Propylene glycol | 2.0 |
| (d) Citric acid monohydrate | 0.05 |
| (e) Sodium citrate dihydrate | 0.26 |
| (f) Sorbic acid | 0.1 |
| (g) Purified water | ad 100.0 |

Manufacturing method (for a batch of 100 kg): Introduce 96.490 kg of g into a dissolutor, add d and e under stirring and continue to stir until dissolution will be complete. Add b and stir until complete dissolution. Add c and f under stirring, then a to the solution and continue to stir until dissolution will be complete. Filter solution through a 0.45 micrometer filter.

### EXAMPLE 15

### Nasal Drop Composition Containing 0.05% (w/w) of Xylometazoline Hydrochloride (with Preservative Sorbic Acid)

| Ingredients | Amount (kg/100 kg) |
|---|---|
| (a) Xylometazoline hydrochloride | 0.05 |
| (b) Chondroitin sulfate (Injectable grade) | 1.0 |
| (c) Propylene glycol | 2.0 |
| (d) Sodium dihydrogen phosphate dihydrate | 0.16 |
| (e) Disodium phosphate dodecahydrate | 0.35 |
| (f) Disodium edetate | 0.05 |
| (g) Sorbic acid | 0.1 |
| (h) Purified water | ad 100.0 |

Manufacturing method (for a batch of 100 kg): Introduce 96.290 kg of h into a dissolutor, add d and e under stirring and continue to stir until dissolution will be complete. Add b and stir until complete dissolution. Add c, f, g under stirring, then a to the solution and continue to stir until dissolution will be complete. Filter solution through a 0.45 micrometer filter. In the following examples 16-18, glycin is added to avoid precipitation of a salt from kationic preservative and sulfate anion.

### EXAMPLE 16

### Nasal Drop Composition Containing 0.1% (w/w) of Xylometazoline Hydrochloride (with Preservative Benzoxonium Chloride)

| Ingredients | Amount (kg/100 kg) |
|---|---|
| (a) Xylometazoline hydrochloride | 0.10 |
| (b) Chondroitin sulfate (Injectable grade) | 1.0 |
| (c) Propylene glycol | 2.0 |
| (d) Glycin | 0.3 |
| (e) Acetic acid 10% | 0.068 |
| (f) Sodium acetate | 0.241 |
| (g) Disodium edetate | 0.05 |
| (h) Benzoxonium chloride | 0.01 |
| (i) Purified water | ad 100.0 |

Manufacturing method (for a batch of 100 kg): Preparation of the solution A: Introduce 91.231 kg of i into a dissolutor, add f and e under stirring, then g and d, and continue to stir until dissolution will be complete. Add b, stir until complete dissolution, then add a and again stir until complete dissolution. Preparation of the solution B: Dissolve h in 5.0 kg of i. Preparation of the final solution: Add the solution B slowly to solution A. Add c under stirring and continue to stir until dissolution will be complete. Filter solution through a 0.45 micrometer filter.

### EXAMPLE 17

### Nasal Drop Composition Containing 0.1% (w/w) of Xylometazoline Hydrochloride (with Preservative Benzalkonium Chloride)

| Ingredients | Amount (kg/100 kg) |
|---|---|
| (a) Xylometazoline hydrochloride | 0.1 |
| (b) Chondroitin sulfate | 1.0 |
| (c) Propylene glycol | 2.0 |
| (d) Glycin | 0.55 |
| (e) Acetic acid 10% | 0.068 |
| (f) Sodium acetate | 0.241 |
| (g) Disodium edetate | 0.05 |
| (h) Benzalkonium chloride | 0.005 |
| (i) Purified water | ad 100.0 |

Manufacturing method (for a batch of 100 kg): Preparation of the solution A: Introduce 90.986 kg of i into a dissolutor, add f and e under stirring, then add g and d, and continue to stir until dissolution will be complete. Add b and a and stir until complete dissolution. Preparation of the solution B: Dissolve h in 5.0 kg of i. Preparation of the final solution: Add the solution B slowly to solution A. Add c under stirring and continue to stir until dissolution will be complete. Filter solution through a 0.45 micrometer filter.

### EXAMPLE 18

### Nasal Spray Composition Containing 0.05% (w/w) of Xylometazoline Hydrochloride (with Preservative Benzalkonium Chloride)

| Ingredients | Amount (kg/100 kg) |
|---|---|
| (a) Xylometazoline hydrochloride | 0.05 |
| (b) Chondroitin sulfate | 1.0 |
| (c) Propylene glycol | 2.0 |
| (d) Glycin | 0.55 |
| (e) Sodium citrate | 0.079 |
| (f) Disodium edetate | 0.05 |
| (g) Benzalkonium chloride | 0.005 |
| (h) Purified water | ad 100.00 |

Manufacturing method (for a batch of 100 kg): Preparation of the solution A: Introduce 91.266 kg of h into a dissolutor, add d, e, f under stirring, and continue to stir until dissolution will be complete. Add b and a and stir until complete dissolution. Preparation of the solution B: Dissolve g in 5.0 kg of h. Preparation of the final solution: Add the solution B slowly to solution A. Add c under stirring and continue to stir until dissolution will be complete. Filter solution through a 0.45 micrometer filter.

1-17. (canceled)

18. A nasal pharmaceutical composition which comprises
(a) at least one active substance suitable for nasal administration, which active substance is selected from the group consisting of xylometazoline, naphazoline, fenoxazoline, oxymetazoline, tetrahydrozoline, tramazoline, phenylephrine, ephedrine, epinephrine, and nasally acceptable salts of any of these compounds,
(b) a mucopolysaccharide which is selected from the group consisting of chondroitin, hyaluronic acid, dermatan, keratan, heparin, acemannan, and nasally acceptable salts of any of said compounds, and (c) propylene glycol.

19. A composition according to claim 18, wherein the active substance (a) is xylometazoline or a nasally acceptable salt thereof.

20. A composition according to claim 18, wherein the mucopolysaccharide (b) is chondroitin sulfate.

21. A composition according to claim 18, wherein propylene glycol (c) is present in an amount of from 0.5 up to 10% (w/w) of the total composition.

22. A composition according to claim 18, wherein propylene glycol (c) is present in an amount of from 1.5 up to 5% (w/w) of the total composition.

23. A composition according to claim 18, wherein the composition further comprises water as vehicle.

24. A composition according claim 18, wherein the composition further comprises a nasally acceptable film-forming agent.

25. A composition according to claim 18, wherein the composition further comprises an essential oil of a plant.

26. A composition according to claim 18, wherein the composition further comprises a nasally acceptable preservative.

27. A composition according to claim 18, wherein the composition is devoid of a nasally acceptable preservative.

28. A nasal pharmaceutical composition which consists essentially of
(a) at least one active substance suitable for nasal administration,
(b) a mucopolysaccharide,
(c) propylene glycol, and
(d) water,
with the proviso that said composition is devoid of fexofenadine and pharmaceutically acceptable salts thereof.

29. A nasal pharmaceutical composition which consists essentially of
(a) at least one active substance suitable for nasal administration,
(b) a mucopolysaccharide,
(c) propylene glycol,
(d) a nasally acceptable preservative, and
(e) water,
with the proviso that said composition is devoid of fexofenadine and pharmaceutically acceptable salts thereof.

30. A composition according to claim 28, wherein the active substance (a) is selected from the group consisting of xylometazoline, naphazoline, fenoxazoline, oxymetazoline, tetrahydrozoline, tramazoline, phenylephrine, ephedrine, epinephrine, and nasally acceptable salts of any of these compounds.

31. A composition according to claim 28, wherein the active substance (a) is xylometazoline or a nasally acceptable salt thereof.

32. A nasal pharmaceutical composition according to claim 28, wherein the mucopolysaccharide (b) is selected from the group consisting of chondroitin, hyaluronic acid, dermatan, keratan, heparin, acemannan, and nasally acceptable salts of any of said compounds.

33. A composition according to claim 32, wherein the mucopolysaccaride (b) is chondrotin sulfate.

34. A composition according to claim 18, wherein the composition is in the form of drops, a solution, a spray or a metered-dose spray.
